(19) United States
(12) Patent Application Publication
(10) Pub. No.: US 2018/0148486 A1
(43) Pub. Date: May 31, 2018

(54) HUMAN CELL LINES MUTANT FOR ZIC2

(71) Applicant: Clark Atlanta University, Atlanta, GA (US)

(72) Inventor: Nathan John Bowen, Atlanta, GA (US)

(21) Appl. No.: 15/578,165

(22) PCT Filed: May 31, 2016
(86) PCT No.: PCT/US2016/035071

§ 371 Date: Nov. 29, 2017

Related U.S. Application Data
(60) Provisional application No. 62/168,024, filed on May 29, 2015.

(51) Int. Cl. C07K 14/47; C12N 15/11; C12N 9/22; C12N 15/85

(52) U.S. Cl. CPC C07K 14/4702; C12N 15/11; C12N 2310/20; C12N 15/85; C12N 2510/02; C12N 9/22

(57) ABSTRACT

Human cell lines mutant for ZIC2 with altered cellular phenotype are disclosed, including HEK 293T, LN prostate cancer, and PC-3 cell lines. Method of making the human cell lines mutant for ZIC2 using gene editing tools such as CRISPR/Cas9 is also disclosed herein. Phenotypic characterization of the clonal mutant lines revealed altered cellular phenotypes relative to the parental lines. For example, ZIC2 protein expression is lost or lowered in these cell lines by western blot analyses. The human cell lines mutant for ZIC2 have various utilities including cancer diagnosis and prognosis.

### CROSS REFERENCE TO RELATED APPLICATIONS

This application claims priority to U.S. provisional patent application Ser. No. 62/168,024, filed on May 29, 2015 to Nathan John Bowen, entitled “Human Cell Lines Mutant for ZIC2” incorporated herein by reference.

### GOVERNMENT RIGHTS

Development of the disclosures described herein was at least partially funded with government support through NIHMD grant number 8G12MD007590, and the U.S. government has certain rights in the inventions.

### STATEMENT REGARDING SEQUENCE LISTING

The Sequence Listing associated with this application is part of the description and is provided in the form of an Annex C/ST.25 text file in lieu of a paper copy, and hereby incorporated by reference into the specification. The name of the text file containing the Sequence Listing is 0309_010_SequenceListing_PatentIn_ST25.txt. The text file is 3 kb, was created on May 31, 2016, and is being submitted electronically via EFS-Web.

### FIELD OF THE INVENTION

The present disclosure is directed to human cell lines mutant for Zic family member 2 (ZIC2) gene. The present disclosure is further directed to ZIC2 mutant proteins derived from human cell lines mutant for ZIC2. Method of making human cell lines mutant for ZIC2 e.g. by CRISPR/Cas9 genome editing is also disclosed.

### BACKGROUND

Prostate cancer is one of the most commonly diagnosed malignancies in American men. A more aggressive form of the disease is particularly prevalent among African Americans. It is also the second leading cause of cancer death in American males, exceeded only by lung cancer. Fortunately, the therapeutic success rate for prostate cancer can be tremendously improved if the disease is diagnosed early. In some cases, when prostate cancer is detected at a very early stage, it can be treated effectively and even eradicated. Consequently, much effort is being placed on detecting prostate cancer in an early, curable stage to decrease the rate of mortality from this disease.

Current advances in molecular techniques have provided new tools facilitating the discovery of new biomarkers for prostate cancer. The National Cancer Institute defines a biomarker as “a biological molecule found in blood, other body fluids, or tissues that is a sign of a normal or abnormal process or of a condition or disease.” A biomarker may be objectively measured and evaluated as an indication of normal biologic processes, pathogenic processes, or pharmacologic responses to a particular treatment or condition. Biomarkers are widely used as analytical tools to assess biological parameters for a rapid and comprehensive therapeutic analysis. In addition, biomarker measures can further the development and evaluation of new therapies for prostate cancer.

For example, screening for elevated prostate-specific antigen (PSA) levels or an abnormal digital rectal exam (DRE), followed by a prostate biopsy, is used to diagnose early stage prostate cancer. Currently, this is the only way to identify prostate cancer in high-risk groups, such as African American men, and to reduce their mortality due to this disease. Diagnosis at an early stage when the cancer is still localized to the prostate gland, leads to nearly a 100% 5-year relative survival rate. In contrast, diagnosis at a later stage, when the cancer has spread to distant lymph nodes, bones or other organs, results in only a 28% 5-year relative survival rate. These data indicate that early diagnosis by routine screening can save lives. However, once diagnosed, not all cancers will progress to late stage disease. Currently, it is difficult to determine which men need treatment and which do not. Approximately 1 in 7 men will be diagnosed with prostate cancer, however only 1 out of 38 will die from prostate cancer. While less than half of the patients are diagnosed with potentially lethal disease (Gleason score>/=7), up to 90% of patients with low-risk disease undergo prostatectomy.

Despite the advances in the discovery of biomarkers for prostate cancer, there remains a need for more clinically reliable biomarkers that will have a high specificity for the diagnosis and prognosis of prostate cancer.

### SUMMARY

Provided herein are cell lines capable of expressing Zic family member 2 (ZIC2) protein or mutant thereof. The cell lines are produced from CRISPR/Cas9 mediated genome editing that comprising co-transfecting host cell lines with a CRISPR/Cas9 plasmid to produce the cell line. The CRISPR/Cas9 plasmid comprises a guide RNA targeting ZIC2 gene. The guide RNA used is capable of guiding CRISPR/Cas9 to ZIC2 gene in the host cells to elicit a double strand break in host gene. Example guide RNA is one of SEQ ID NO. 2-SEQ ID NO. 6 or a functional variant thereof. The host cell lines can be any host cell that is capable of receiving recombinant vectors. Example host cell lines include HEK 293T, LNcaP, and PC-3. The HEK 293T cell line bearing parental or wild type ZIC mutation is a triploid in the region of ZIC2, each of the three sequences of the triploid having a sequence over the region of the genome near the 118 sgRNA specified by SEQ ID NO. 9. In one embodiment the HEK 293T cell line mutant ZIC2 is a triploid in the region of ZIC2, having the three sequences of the triploid each over the region of the genome near the 118 sgRNA specified by SEQ ID NO. 7, 8, and 10.

In one aspect, a method of producing a cell line capable of expressing Zic family member 2 (ZIC2) protein or mutant thereof using CRISPR/Cas9 mediated genome editing is disclosed. The method comprises co-transfecting host cell lines with a CRISPR/Cas9 plasmid to produce the cell line. The CRISPR/Cas9 plasmid comprises a guide RNA targeting ZIC2 gene. The guide RNA used is capable of guiding CRISPR/Cas9 to ZIC2 gene in the host cells to elicit a double strand break in host gene. Example guide RNA is one of SEQ ID NO. 2-SEQ ID NO. 6 or a functional variant thereof. The host cell lines can be any host cell that is capable of receiving recombinant vectors. Example host cell lines include HEK 293T, LNcaP, and PC-3. In one embodiment the method comprises inserting a guide RNA of SEQ ID NO. 2 or 6 or a functional variant thereof. In one embodiment, the method comprises co-transfecting a HEK 293T host cell line. In one embodiment, the method further comprising isolating ZIC2 protein from one of the ZIC2 mutant cell lines disclosed herein.

### BRIEF DESCRIPTION OF THE DRAWINGS

FIG. 1 is a schematic diagram illustration of the gene editing process using CRISPR/Cas9 systems.

FIG. 2 is a schematic diagram illustrating an example plasmid map for targeted ZIC2 knockout using CRISPR/Cas9 systems.

FIG. 3 shows RT-PCR results of mRNA ZIC expression in various prostate cancer cell lines.

FIG. 4 shows the overexpression of MYC-tagged ZIC2 were detected using antibodies.

FIG. 5 shows the endogenous expression of ZIC2 protein in various prostate cancer cell lines.

FIG. 6 shows the positive results obtained from immunohistochemistry (IHC) on prostate cancer tissues with Abcam RabMab.

FIG. 7 shows the western blot of multiple ZIC2 targeting shRNA expressing cell lines established in HEK 293T.

FIG. 8A is light microscopy image of parental HEK293T.

FIG. 8B is light microscopy image of HEK293T mutant HM1.

FIG. 8C is light microscopy image of HEK293T mutant HM3.

FIG. 8D is light microscopy image of HEK293T mutant HM4.

FIG. 8D is light microscopy image of HEK293T mutant HM5.

FIG. 8F is light microscopy image of parental LN prostate cancer.

FIG. 8G is light microscopy image of LN prostate cancer mutant LM1.

FIG. 8H is light microscopy image of parental PC-3.

FIG. 8I is light microscopy image of PC-3 mutant PM1.

FIG. 9A shows results from Western Blot analysis carried out on parental HEK 293T cell lines.

FIG. 9B shows results from Western Blot analysis carried out on individual putative mutant lines.

FIG. 9C shows results from PCR analysis of ZIC2 in parental or wild type HEK 293T cell lines and two mutants of HEK 293T, A7 and F8.

FIG. 9D shows results from Western Blot analysis of parental LN prostate cancer (LNCaP) and mutant of LNCaP LM1.

FIG. 9E shows results from Western Blot analysis of parental PC-3 and mutants of PC-3 PM1 and PM2.

FIG. 9F shows results from PCR analysis of ZIC2 in parental or wild type PC-3 cell lines and two mutants of PC-3, PM1 and PM2.

FIG. 10 shows the DNA sequence of ZIC2 over the region of the genome near the 118 sgRNA in parental compared with the triploid sequences of F8.

### DETAILED DESCRIPTION

The present disclosure may be understood more readily by reference to the following detailed description of embodiments and to the Figures and their previous and following description.

In this specification and the appended claims, the singular forms “a,” “an” and “the” include plural reference unless the context clearly dictates otherwise.

The term “cell” is used in its usual biological sense, and does not refer to an entire multicellular organism. The cell can, for example, be in vitro, e.g., in cell culture.

The term “host cell” includes an individual cell or cell culture which can be or has been a recipient of any virus or recombinant vector(s). Host cells include progeny of a single host cell, and the progeny may not necessarily be completely identical (in morphology or in total DNA complement) to the original parent cell due to natural, accidental, or deliberate mutation and/or change. A host cell includes cells infected or transfected with a virus or recombinant vector.

The cells of disclosed herein are grown in any vessel, flask, tissue culture dish or device used for culturing cells that provides a suitable surface for cell attachment and spreading (e.g., Culture of Hematopoietic Cells (Culture of Specialized Cells) R. I. Freshney et al., Ed., I. Freshney; Wiley-Liss 1994; incorporated by reference herein). As used herein, the term “attachment” refers to cell adherence and spreading on a surface in such a device, where factors promoting cell attachment and spreading directly contact the cultured cells, Cell growth is maintained directly on surfaces of the culture vessel or on supplemental inserts such as cartridges or membranes placed within the vessel, Appropriate attachment and spreading surfaces are produced either by initially selecting a suitable surface material or by subsequently treating an existing surface. Common treatments are well known and include coating surfaces with compositions that promote attachment and spreading. Such compositions are also well known and include polybasic amino acids such as polyomithine and polylysine. Furthermore, the attachment surface may be coated or provided with a known extracellular matrix protein or with compositions or artificial environments that are functionally equivalent to an in vivo extracellular matrix. Typical cell matrix compositions are well known and include laminin, collagen and fibronectin. Other extra cellular matrix proteins or artificial extracellular matrix environments, which mimic an in vivo extracellular matrix, are known in the art (see e.g., Synthetic Biodegradable Polymer Scaffolds (Tissue Engineering) A. Atala and D. J. Mooney (Eds.) Birkhauser, 1997).

The term “confluence” as used herein, refers to a density of cultured cells in which the cells contact one another covering most or all of the surfaces available for growth.

During pre-confluent growth, selected cells behave like regenerating hepatocytes demonstrating corresponding patterns of regulation of gene expression.

A cell line is a population or mixture of cells of common origin growing together after several passages in vitro. By growing together in the same medium and culture conditions, the cells of the cell line share the characteristics of generally similar growth rates, temperature, gas phase, nutritional and surface requirements. An enriched cell line is one in which cells having a certain trait, e.g. ZIC2 knockout, are present in greater proportion after one or more subculture steps, than the original cell line.

Clonal cells are those which are descended from a single cell. As a practical matter, it is difficult to obtain pure cloned cell cultures of mammalian cells. A high degree of cell purity can be obtained by successive rounds of cell enrichment. As used herein, a cell culture in which at least 90% of the cells possess a defined set of traits is termed a cloned cell culture.

Along with Cas9 nuclease, CRISPR experiments require the introduction of a guide RNA (sgRNA) containing an approximately 20 base sequence specific to the target DNA 5′ of a non-variable scaffold sequence. sgRNA can be delivered as RNA or by transforming with a plasmid with the sgRNA-coding sequence under a promoter. The terms guide RNA or sgRNA herein refers the RNAs used in CRISPER experiments.

General

Clinical decision-making, with respect to the course of treatment for prostate cancer (prostate cancer), is in need of biological markers that 1) predict which cancers are indolent, and therefore don't need treatment, 2) in non-indolent cancers, which ones can be treated successfully with a localized treatment and 3) which patients are likely to have an aggressive form of cancer that will require more aggressive surgical and chemotherapeutic treatments.

Candidate genes identified as being aberrantly over-expressed in prostate cancer and has functional significance at the protein level is the gene encoding the C2H2 zinc finger containing transcription factor, Zic family member 2 (ZIC2). In addition to prostate cancer, high levels of ZIC2 gene expression are significantly associated with a lower disease-free survival and lower overall survival rates in oral squamous cell carcinomas (OSCCs). Because ZIC2 mRNA has been shown to be expressed in human and mouse embryonic stem cells, it is believed to play a role in unlimited self-renewal and pluripotency. Therefore, ZIC2 represent a useful biomarkers for disease detection and progression in prostate cancer. For example, the ability to detect ZIC2 protein levels in local biopsy samples may indicate the likelihood of metastatic potential.

ZIC2 belongs to a family of zinc finger transcription factors, which represent the vertebrate homologues of the Drosophila pair rule gene odd-paired. There are 5 ZIC paralogues in the human genome, ZIC1-5 that are all thought to be DNA binding transcription factors. The acronym ZIC comes from an earlier name for the genes, Zinc finger protein of the cerebellum. ZIC2 is expressed during normal development and tissue homeostasis in neural crest and mesoderm development and in the adult cerebellum of vertebrates. In humans, haplo insufficiency of ZIC2 results in the neural tube closure defect, holoprosencephaly, characterized by a single-lobed brain structure and severe skull and facial defects. Homozygous knock out or complete loss of function in mice leads to early embryonic lethality. Only heterozygous or knock down animals are born alive and most display neural tube closure defects. These studies indicated that ZIC2 is necessary for neural plate expansion by promoting neural ectodermal precursor and neural stem cell proliferation and delaying the establishment of differentiating neural progenitors.

In addition, ZIC2 is thought to belong to an extended transcriptional network involved in maintaining pluripotency in embryonic stem cells by interacting directly with the pluripotency factor OCT4/POU5F1. The expression of ZIC2 in neurons in the cerebellum, which are extremely long-lived cells, and in embryonic stem cells suggests ZIC2 may play a role in cell immortalization, a characteristic of both stems cells and cancer. Expression of ZIC2 is believed to play a similar role in the immortalization, proliferation and the lack of differentiation that is characteristic of prostate cancer cells.

Genetic Editing with CRISPR/Cas9

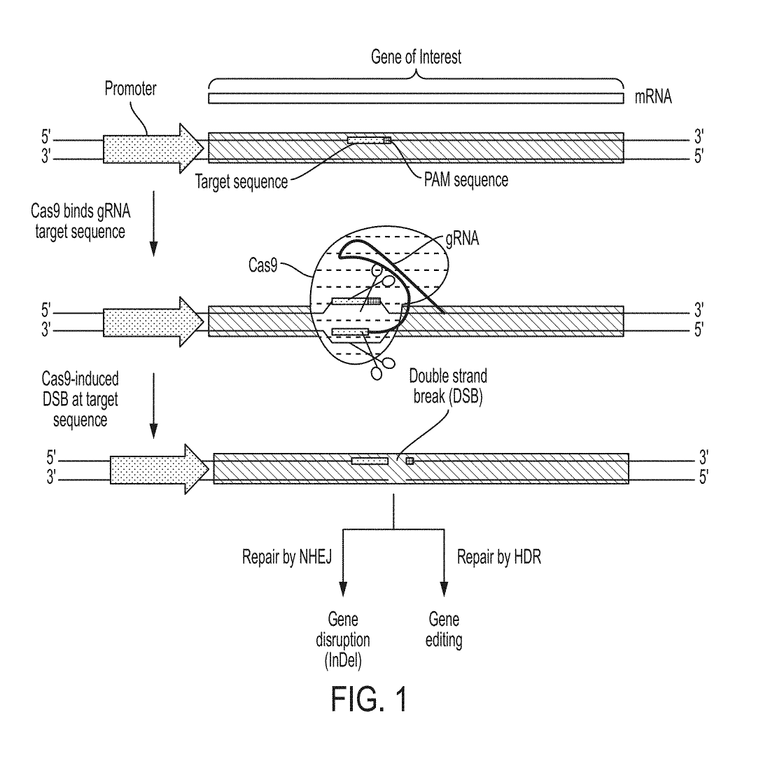

Cas9 is a nuclease that was first discovered as a component of the CRISPR system in Streptococcus pyogenes and has been adapted for utility in mammalian cells. RNA-guided Cas9 is able to efficiently introduce precise double-stranded breaks at endogenous genomic loci in mammalian cells with high efficiencies. The gene editing process using CRISPR/Cas9 system is illustrated in FIG. 1. Cas9 nucleases can be directed by short guide RNAs (sgRNA) to induce precise cleavage at endogenous genomic loci in human and mouse cells. Cas9 can also be converted into a nicking enzyme to facilitate homology-directed repair with minimal mutagenic activity. Lastly, multiple guide sequences can be encoded into a single CRISPR array to enable simultaneous editing of several sites within the mammalian genome, demonstrating easy programmability and wide applicability of the RNA-guided nuclease technology. Cas9 vectors express the Cas9 nuclease and the RNA sequences that guide the nuclease to its genomic target. Cas9 expression is driven by a choice of promoters and can be monitored by linked expression of green or red fluorescent proteins.

CRISPR/Cas9 mediated genome editing method disclosed herein comprises inserting a guide RNA into a ZIC2 gene with CRISPR/Cas9 mediated genome editing to produce a plasmid. The cell lines are then subsequently produced using a host cell lines co-transfected with the plasmid produced by CRISPR/Cas9 mediated genome editing. The guide RNA used is capable of guiding CRISPR/Cas9 to the double strand break of host gene. Example guide RNA is one of SEQ ID NO. 1-SEQ ID NO. 6 or a functional variant thereof. The host cell lines can be any host cell that is capable of receiving recombinant vectors. Example host cell lines include HEK 293T, LNcaP, and PC-3.

Plasmids

The nucleic acids that are delivered to cells typically contain expression controlling systems. For example, the inserted genes in viral and retroviral systems usually contain promoters, and/or enhancers to help control the expression of the desired gene product. A promoter is generally a sequence or sequences of DNA that function when in a relatively fixed location in regard to the transcription start site. A promoter contains core elements required for basic interaction of RNA polymerase and transcription factors, and may contain upstream elements and response elements.

Promoters controlling transcription from vectors in mammalian host cells may be obtained from various sources, for example, the genomes of viruses such as: polyoma, Simian Virus 40 (SV40), adenovirus, retroviruses, hepatitis-B virus and cytomegalovirus (CMV), or from heterologous mammalian promoters, e.g. beta actin promoter. The early and late promoters of the SV40 virus are conveniently obtained as an SV40 restriction fragment which also contains the SV40 viral origin of replication. The immediate early promoter of the human cytomegalovirus is conveniently obtained as a HindIII E restriction fragment. For instance, promoters from the host cell or related species also are useful herein.

Enhancer generally refers to a sequence of DNA that functions at no fixed distance from the transcription start site and can be either 5′ or 3′ to the transcription unit. Furthermore, enhancers can be within an intron as well as within the coding sequence itself. They are usually between 10 and 300 by in length, and they function in cis. Enhancers function to increase transcription from nearby promoters. Enhancers also often contain response elements that mediate the regulation of transcription. Promoters can also contain response elements that mediate the regulation of transcription. Enhancers often determine the regulation of expression of a gene. While many enhancer sequences are now known from mammalian genes (globin, elastase, albumin, -fetoprotein and insulin), typically one will use an enhancer from a eukaryotic cell virus for general expression. Examples are the SV40 enhancer on the late side of the replication origin (bp 100-270), the cytomegalovirus early promoter enhancer, the polyoma enhancer on the late side of the replication origin, and adenovirus enhancers.

The promoter and/or enhancer may be specifically activated either by light or specific chemical events which trigger their function. Systems can be regulated by reagents such as tetracycline and dexamethasone. There are also ways to enhance viral vector gene expression by exposure to irradiation, such as gamma irradiation, or alkylating chemotherapy drugs.

In certain embodiments the promoter and/or enhancer region can act as a constitutive promoter and/or enhancer to maximize expression of the region of the transcription unit to be transcribed. In certain constructs the promoter and/or enhancer region are active in all eukaryotic cell types, even if it is only expressed in a particular type of cell at a particular time. A promoter of this type for example is the CMV promoter (650 bases). Other example promoters are SV40 promoters, cytomegalovirus (full length promoter), and retroviral vector LTR.

Expression vectors used in eukaryotic host cells (yeast, fungi, insect, plant, animal, human or nucleated cells) may also contain sequences necessary for the termination of transcription which may affect mRNA expression. These regions are transcribed as polyadenylated segments in the untranslated portion of the mRNA encoding tissue factor protein. The 3′ untranslated regions also include transcription termination sites. In some embodiments, the transcription unit also contain a polyadenylation region. One benefit of this region is that it increases the likelihood that the transcribed unit will be processed and transported like mRNA. The identification and use of polyadenylation signals in expression constructs is well established. For instance, homologous polyadenylation signals can be used in the transgene constructs. In certain transcription units, the polyadenylation region is derived from the SV40 early polyadenylation signal and consists of about 400 bases. In one embodiment, the polyadenylation signal is derived from the human growth hormone poly-A signal (hGH-pA). The transcribed units can contain other standard sequences alone or in combination with the above sequences improve expression from, or stability of, the construct.

The expression vectors can include nucleic acid sequence encoding a marker product. This marker product is used to determine if the gene has been delivered to the cell and once delivered is being expressed. For example, the marker genes can be the E. Coli lacZ gene, which encodes β-galactosidase, and green fluorescent protein.

In some embodiments the marker may be a selectable marker. Examples of suitable selectable markers for mammalian cells are dihydrofolate reductase (DHFR), thymidine kinase, neomycin, neomycin analog G418, hydromycin, and puromycin. When such selectable markers are successfully transferred into a mammalian host cell, the transformed mammalian host cell can survive if placed under selective pressure. There are two widely used distinct categories of selective regimes. The first category is based on a cell's metabolism and the use of a mutant cell line which lacks the ability to grow independent of a supplemented media. Two examples are: CHO DHFR-cells and mouse LTK-cells. These cells lack the ability to grow without the addition of nutrients such as thymidine or hypoxanthine. Because these cells lack certain genes necessary for a complete nucleotide synthesis pathway, they cannot survive unless the missing nucleotides are provided in a supplemented media. An alternative to supplementing the media is to introduce an intact DHFR or TK gene into cells lacking the respective genes, thus altering their growth requirements. Individual cells which were not transformed with the DHFR or TK gene will not be capable of survival in non-supplemented media.

The second category is dominant selection which refers to a selection scheme used in any cell type and does not require the use of a mutant cell line. These schemes typically use a drug to arrest growth of a host cell. Those cells which have a novel gene would express a protein conveying drug resistance and would survive the selection. Examples of such dominant selection use the drugs neomycin, mycophenolic acid, or hygromycin. The three examples employ bacterial genes under eukaryotic control to convey resistance to the appropriate drug G418 or neomycin (geneticin), xgpt (mycophenolic acid) or hygromycin, respectively. Others include the neomycin analog G418 and puramycin.

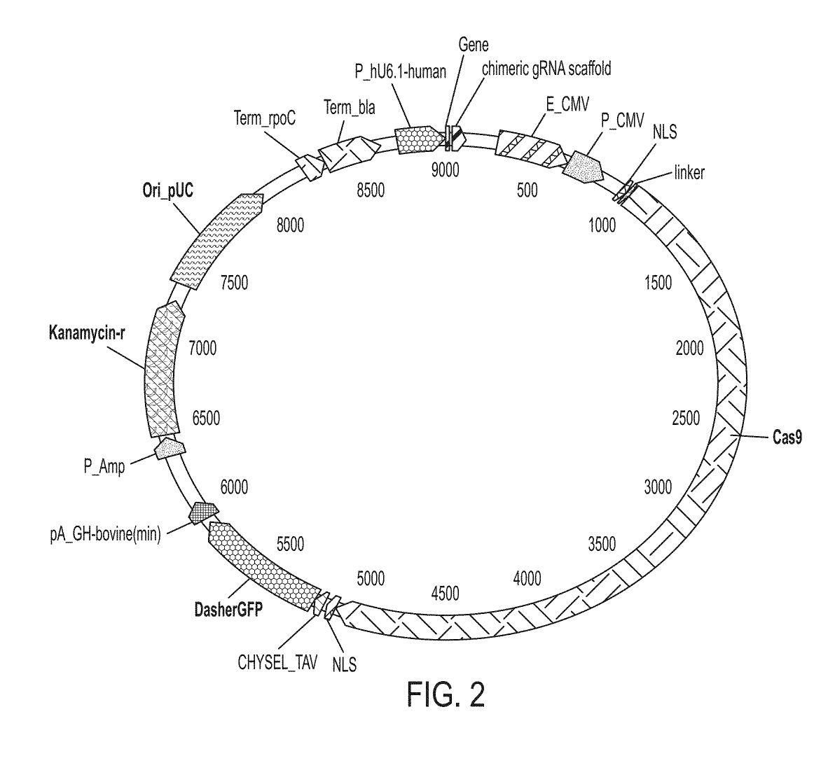

An example plasmid map for targeted ZIC2 knockout using CRISPR/Cas9 systems is shown in FIG. 2. Specifically, the chimeric guide RNA (gRNA) scaffold consists of a 20-nt target specific complementary region, a 42-nt Cas9-binding RNA structure and a 40-nt transcription terminator derived from S. pyogenes that directs Cas9 nuclease to the target site for genome modification. The TAV CHYSEL (cis-acting hydrolase element) or 2A peptide was found in the insect Thosea asigna virus (TaV). Multiple proteins can be efficiently produced from one coded peptide by a mechanism that relies on the self-cleaving 2A peptide sequence which allows for translational ‘skipping’. The 2A peptide mediates the co-translational cleavage of a polyprotein. After cleavage, the short 2A peptide remains fused to the C terminus of the ‘upstream’ protein, while a proline is added to the N-terminus of the ‘downstream’ protein. DasherGFP green fluorescent reporter protein that is used as a selectable marker for expression monitoring of target protein with Ex/Em of 505/525 nm. The cytomegalovirus enhancer element (E CMV) plays a critical role in overcoming inefficient transcriptional activities of promoters, thereby enhancing transcription. The hCMV IE1 enhancer/promoter is one of the strongest enhancer/promoters known and is active in a wide range of cell types. Kanamycin-r is an effective bacteriocidal agent that inhibits ribosomal translocation thereby causing miscoding. The gene coding for kanamycin resistance is Neomycin phosphotransferase II (NPT II/Neo). E. coli transformed with plasmid containing the kanamycin resistance gene can grow on media containing 50-100 μg/ml kanamycin. Kanamycin is a white to off-white powder that is soluble in water (50 mg/ml). A nuclear localization signal (NLS) is an amino acid sequence that ‘tags’ a protein for import into the cell nucleus by nuclear transport. Typically, this signal consists of one or more short sequences of positively charged lysines or arginines exposed on the protein surface. Chelsky et al. proposed the consensus sequence K-K/R-X-K/R for monopartite NLSs. A protein translated with a NLS will bind strongly to importin, and together the complex will move through the nuclear pore. Once in the nucleus, Ran-GTP binds to the importin-protein complex, causing importin to lose affinity for the protein, thereby releasing the protein. The origin of replication is a sequence in a genome at which replication is initiated. The Ori_pUC is a mutated form of origin derived from E. coli plasmid pBR322 which allows production of greater than 500 copies of plasmid per cell. The CMV promoter (P_CMV) is a constitutive mammalian promoter and mediates strong expression in various cellular systems. Strong expression in HEK 293 and CHO cells have been observed. CMV mediates strong Cas9 transient expression compared to CAG or CBh promoters. CMV promoter mediated only transient expression in hESCs. CMV promoters have been reported to be prone to ‘silencing’ in some cell lines. P_hU6.1-human is a type 3 core promoter for RNA expression that was originally identified in mammalian U6 snRNA genes, which encode the U6 snRNA component of the spliceosome. A hallmark of type 3 is the presence of a TATA box. And the bovine growth hormone polyadenylation (bgh-PolyA) signal (pA_GHbovine (min)) is a specialized termination sequence for protein expression in eukaryotic cells.

Host Cell Lines

The host cells lines contemplated herein include eukaryotic cell lines suitable for producing mutant for ZIC2 disclosed herein. Contemplated cells include Hek293 and multiple prostate cancer cell lines including, PrEC, RWPE1, RWPE1-ZIC2, INCaP, C33, C42, C81, DU145, PC-3, PC3M, E006AA, MDA PCA2A, and MDA PCA2B.

Human Cell Lines Mutant for ZIC2

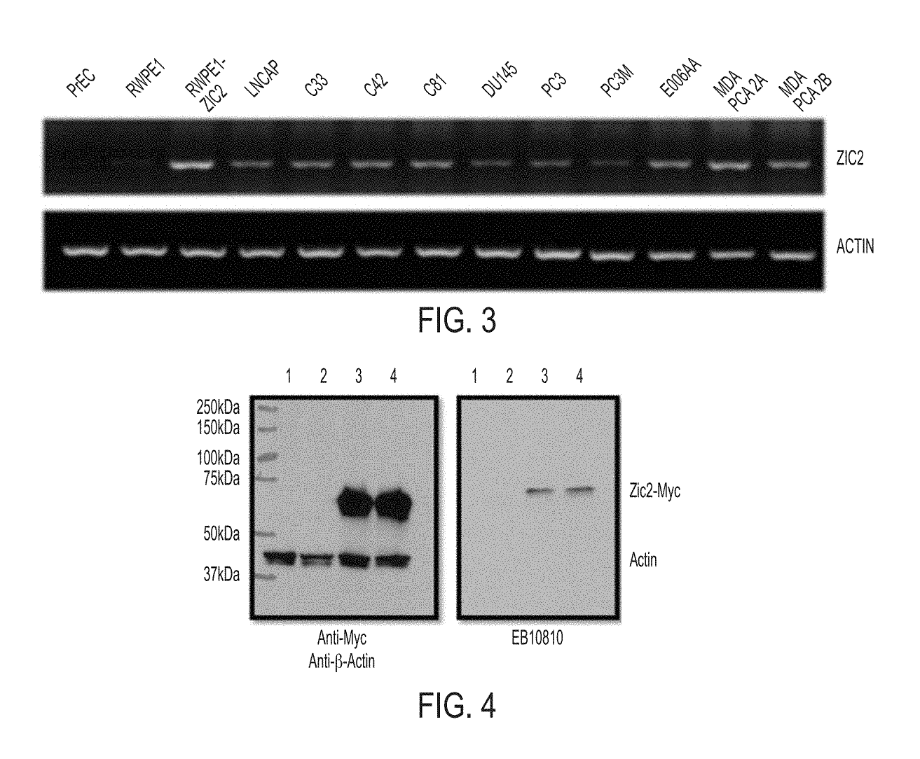

ZIC2 mRNA is expressed in multiple prostate cancer cell lines. RT-PCR of mRNA ZIC expression in various prostate cancer cell lines is shown in FIG. 3. The CRISPR/Cas9 genome editing system allows complete gene knockout (KO) as opposed to partial gene knockdown (KD) by RNA interference. ZIC2 sgRNA-containing CRISPR/Cas9 genome editing plasmids for example plasmids from Horizon Discovery Group, Cambridge, UK and DNA 2.0, Menlo Park, Calif. are co-transfected followed by establishing clonal ZIC2 mutant lines and matched isogenic parental lines using Fluorescence Activated Cell Sorting (FACS) of single cells into 96 well plate for long-term cell culture. ZIC2 in the produced cell lines are then subjected to Western Blot Analysis.

HEK293T, LN prostate cancer and PC-3 cell lines were edited using CRISPR/Cas9 for ZIC2 genomic editing. Phenotypic characterization of the clonal mutant lines revealed altered cellular phenotypes, relative to the parental lines, consistent with a mesenchymal to epithelial transition (MET) in HEK293T and PC-3 lines and an epithelial to mesenchymal transition (EMT) in the LN prostate cancer line.

The examples below serve to further illustrate the invention, to provide those of ordinary skill in the art with a complete disclosure and description of how the compounds, compositions, articles, devices, and/or methods claimed herein are made and evaluated, and are not intended to limit the scope of the invention. In the examples, unless expressly stated otherwise, amounts and percentages are by weight, temperature is in degrees Celsius or is at ambient temperature, and pressure is at or near atmospheric.

### EXAMPLES

### Example 1 Detection of ZIC2 Protein in Cell Lines

After multiple attempts, endogenous ZIC2 protein in cell lines were not detected with many (>5) commercially available rabbit polyclonal and mouse monoclonal antibodies (data not shown). The overexpression of a MYC-tagged ZIC2 were detected with these antibodies as shown in FIG. 4. Specifically, Mock-transfected RWPE1 in lanes 1 and expressing GFP in lanes 2. Lanes 3 and 4 are RWPE1 lysate (60 micro gram protein in RIPA buffer) overexpressing Human ZIC2 with C-terminal MYC tag probed with anti-myc Tag ( 1/1000) and anti-beta-Actin in the left panel and probed with EB10810 anti-ZIC2 (0.5 micro gram/mL in the right panel. Primary incubations were overnight at 4° C. and detected by chemiluminescence. The sensitivity of tests was extremely weak indicating they were likely unable to detect endogenous ZIC2 protein if it was indeed present.

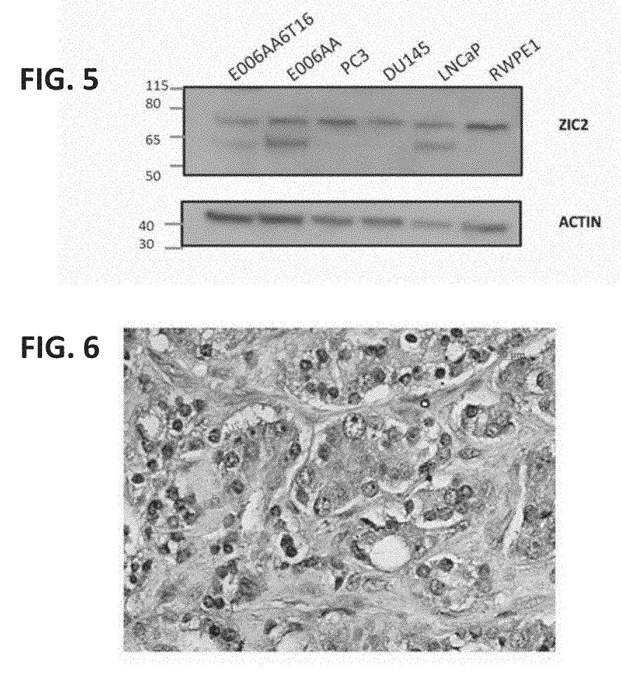

Subsequently a commercial rabbit monoclonal antibody (RabMab, Abcam, clone EPR7790, ab150404) was identified that generated against a region of ZIC2 from humans spanning the amino acids from 100-130 that yields consistently positive results in most all cell lines tested. This antibody is extremely sensitive (used at 1/10,000 in primary antibody dilutions) indicating that it can detect the potentially low abundance ZIC2 protein. This antibody consistently detects two bands just above and below the 65KD molecular marker as shown in FIG. 5. It appears the lower band is absent in the androgen receptor (AR) negative cell lines PC-3 and AR. Immunohistochemistry (IHC) on prostate cancer tissues with Abcam RabMab has produced positive results as shown in FIG. 6.

### Example 2 ZIC2 Targeting shRNA Failed to Produce Mutant ZIC2

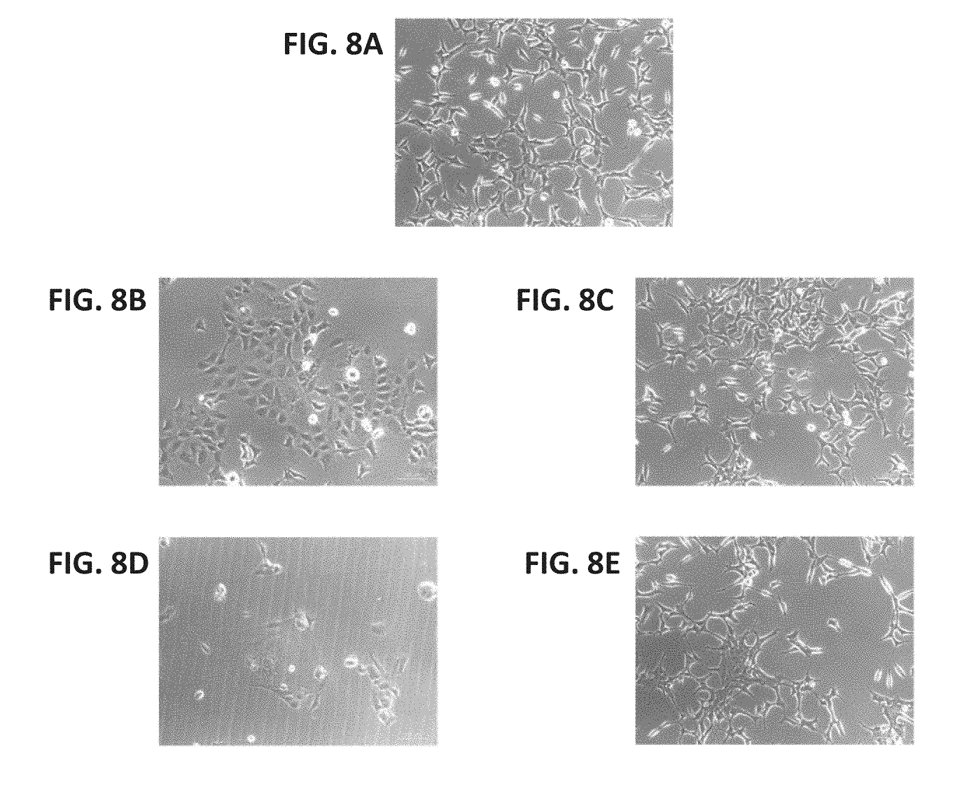

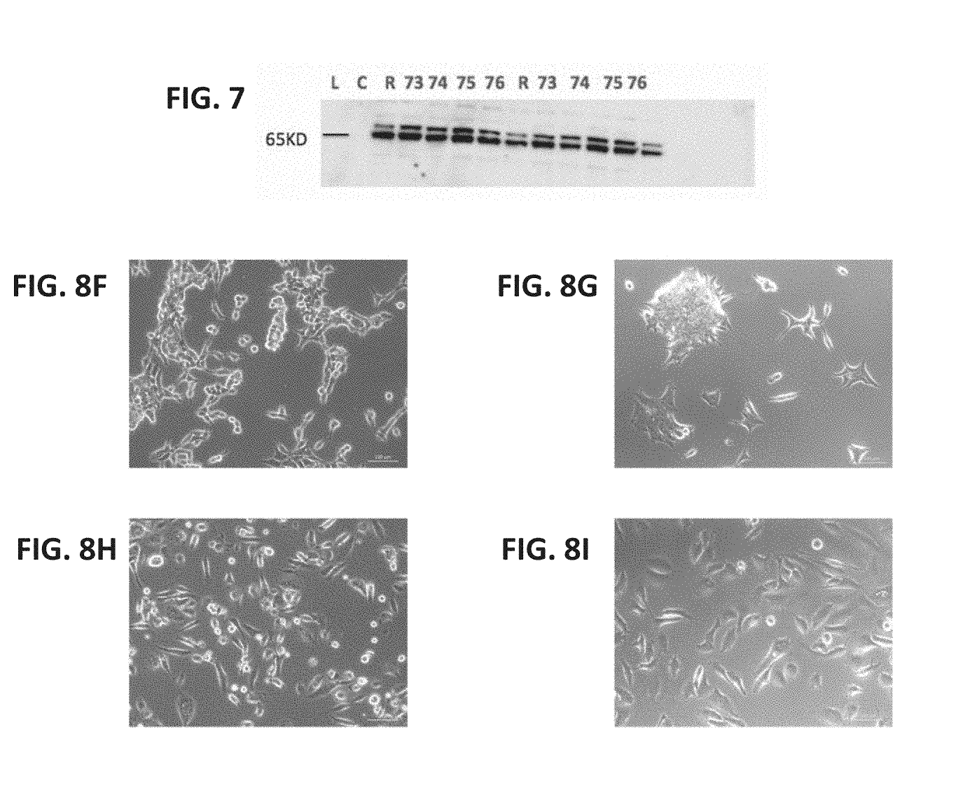

To verify the specificity of the commercial antibodies for ZIC2, multiple stable ZIC2 targeting shRNA expressing cell lines were established in the HEK293T cell line. Recent evidence suggests that HEK293T cells are neuronal in origin and may fortuitously be good candidates for endogenous ZIC2 expression). Western blot revealed little to no down-regulation of ZIC2 in all of the lines generated as shown in FIG. 7. Specifically, C lane is no shRNA, R lanes are scrambled shRNA, and numbered lanes 73-76 represent individual lines of ZIC2 shRNA transfected and stably selected lines. The characteristic two bands flanking the 65 KD marker are present in HEK293T (the same size bands as detected in the prostate cancer cell lines in FIG. 4) and appear mostly unaltered in these stable lines. These results indicated that either: (1) the targeting plasmids used were ineffective; (2) that loss of ZIC2 may be lethal to cell lines or (3) at least, the down-regulation of ZIC2 protein levels is difficult to achieve by this method.

### Example 3 Human Cell Lines Mutant ZIC2 Production and Characterization

Cell line knock outs of ZIC2 have been produced using the CRISPR/Cas9 gene editing method.

A set of five CRISPR/Cas9/GFP encoding targeting plasmids (SEQ ID NO: 2-6) with guide RNAs (gRNAs) against the first exon of ZIC2 as shown in Table 1 were acquired from Horizon Discovery.

TABLE 1
Guide RNA Sequences

| | Gene | Guide ID | Guide Sequence |
|---|---|---|---|
| SEQ ID NO: 2 | ZIC2 | 114114 | CGGACCCGCGTCCAGGAGCA |
| SEQ ID NO: 3 | ZIC2 | 114115 | CCGCGGCGGAGTGGTGATGG |
| SEQ ID NO: 4 | ZIC2 | 114116 | TTCACGGTCCTGCATCTCGG |
| SEQ ID NO: 5 | ZIC2 | 114117 | AGCCTGGCGGCGGCGCAGAA |
| SEQ ID NO: 6 | ZIC2 | 114118 | GAAGGCTCCCATGTGCGCGG |

CRISPR/Cas9 mediated genome editing was performed on HEK 293T cell lines (ATCC® CRL-3216™) to induce a frame-shift mutation in the 5′ region of the first exon of Zic family member 2 (ZIC2). In the GRCh37/hg19 release of the human genome, the coding region of ZIC2 is located at the chromosomal coordinates of 100,634,319-100,637,936 of chromosome 13. Briefly, guide RNAs corresponding to 5′-CGGACCCGCGTCCAGGAGCA-3′ (SEQ ID NO:2) and 5′-GAAGGCTCCCATGTGCGCGG-3′ (SEQ ID NO:6) were each cloned into the DNA2.0 plasmid pD1301-AD. The plasmids were co-transfected into HEK 293T cell lines with FuGENE® 6 reagent. Twenty-four hours following transfection, cells were sorted into 96-well tissue culture plates using a BD FACSJazz™ cell sorter. Plates were incubated at 37° C./5% CO₂ and monitored daily for several weeks until growth of cells was observed. Clones were transferred to 6-well tissue culture plates and cultured to confluence. Cells were then transferred to T-75 flasks for continued growth. The cell lines were subjected to Western Blot analysis for ZIC2 protein detection. Light microscopy images of mutant cell lines in culture were obtained for phenotype comparison against parental cell lines. The phenotypic characterization of the cell lines are shown in FIGS. 8A-8I. The parental cell lines were not subjected to co-transfections with CRISPR/Cas9 genome editing plasmids containing sgRNAs targeted against ZIC2. Images are shown at 100× magnification. Specifically, FIG. 8A is image of parental HEK293T and FIGS. 8B-8E are images of HEK293T mutants HM1, HM3, HM4, and HM5 respectively. FIG. 8F is image of parental LN prostate cancer and FIG. 8G is an image of LN prostate cancer (LNCaP) mutant LM1. FIG. 8H is image of parental PC-3 and FIG. 8I is an image of PC-3 mutant PM1.

Western blot analysis of HEK293T, LN prostate cancer (LNCaP), and (C) PC-3 cell lines post-FACS are obtained using anti-ZIC2 antibody (abcam, 1:10,000) and anti-ACTB antibody (Millipore, 1:1,000). The ACTB (actin, beta) is used for loading control.

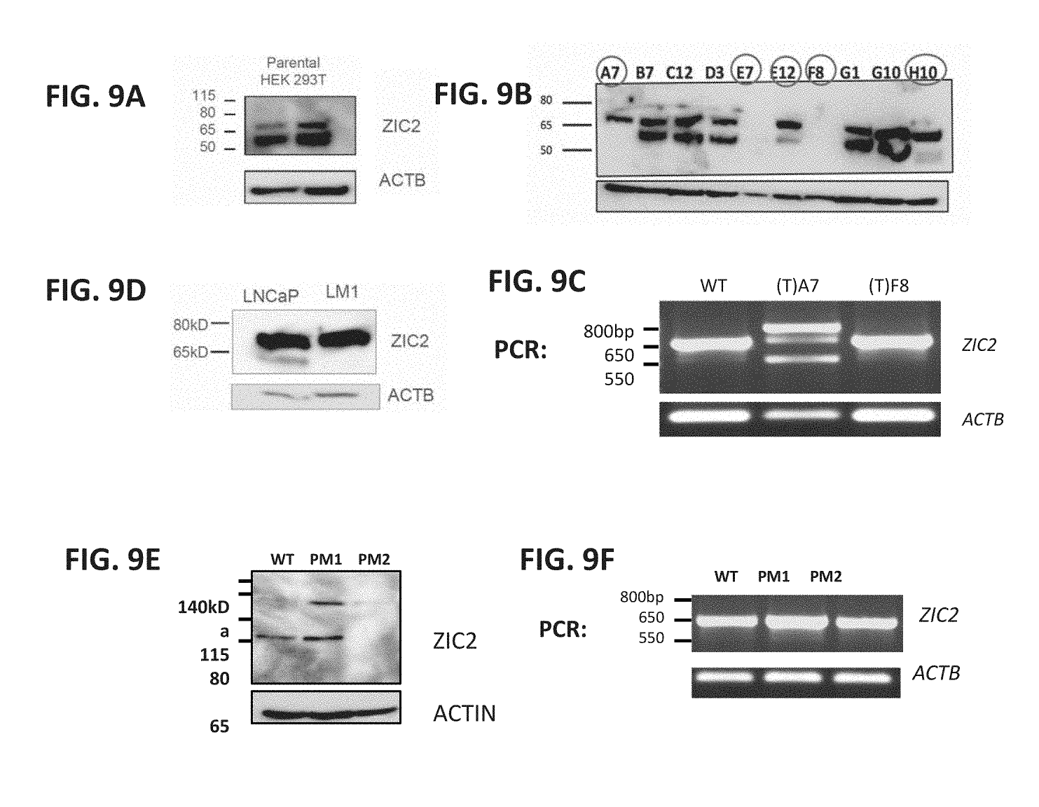

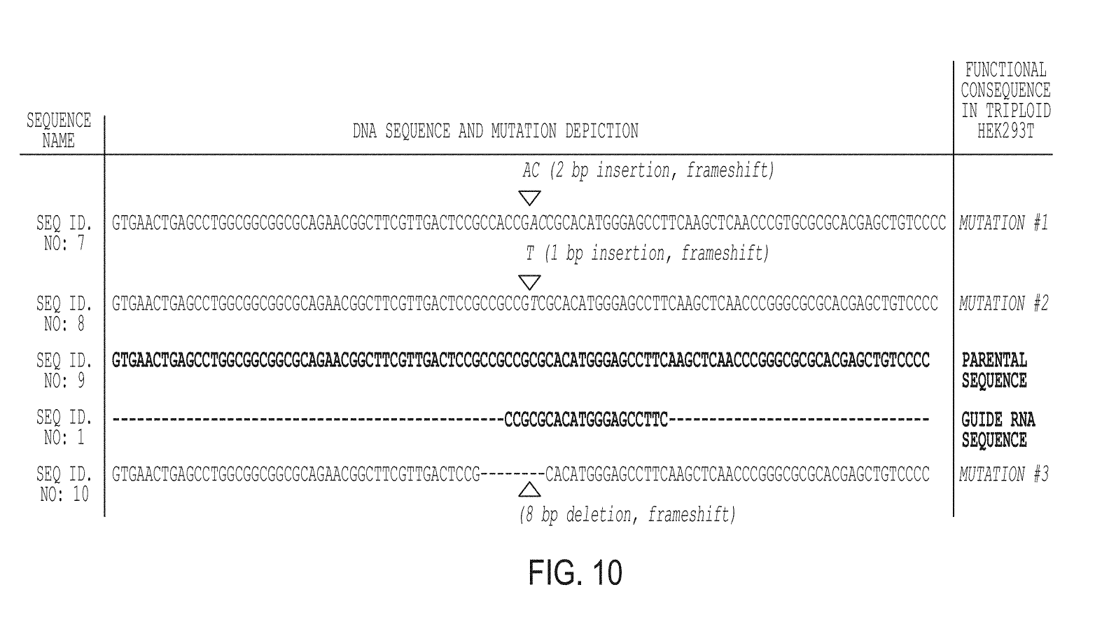

Using HEK293T as host cell line, cloned cell lines A7, B7, C12, D3, E7, E12, F8, G1, G10 and H10 were obtained. The cell lines were subjected to Western Blot analysis for ZIC2 protein detection and the results are shown in FIG. 9A and FIG. 9B. The western blots carried out on parental HEK 293T cell lines is shown in FIG. 9A and individual putative mutant lines are shown in FIG. 9B. PCR analysis of A7 and F8 mutants against wild type or parental are obtained and shown in FIG. 9C. The lower panels contain Westerns detecting Actin, beta (ACTB) for loading controls. It appears that HEK293T cells are triploid (meaning they have 3 chromosome equivalents of this region) over this region of ZIC2. Sample cell line A7 appears to be a double mutant and F8 appears to be a triple mutant. The DNA sequence of ZIC2 over the region of the genome near the 118 sgRNA in parental compared with the triploid sequences of F8 are shown in FIG. 10, indicating the 3 mutations in each F8 triploid sequences. Specifically, a first sequence of mutant F8 has mutation #1 characterized with 2 base pair insertion and frameshift. A second sequence of mutant F8 has mutation #2 characterized with 1 base pair insertion and frameshift. A third sequence of mutant F8 has mutation #3 characterized with 8 base pair deletion and frameshift.

Western Blot analysis of parental LN prostate cancer and mutant LM1 is shown in FIG. 9D. Western Blot analysis of parental PC-3 and mutants PM1 and PM2 is shown in FIG. 9E. PCR analysis of parental PC-3 and mutants PM1 and PM2 is shown in FIG. 9F. PC-3 mutant 1 (PM3) has more ZIC2 and PC-3 mutant 2 (PM2) has less ZIC3 than the parental. It appears that PM 1 is heterozygous and PM2 is homozygous.

Western blot analysis of these cloned cell lines of HEK293T indicate that at least the lower of the two bands is likely ZIC2, since it disappears in 3/10 lines. Two of the ten lines show potential loss of both bands. Finally 5/10 lines appear unmutagenized or wild-type as they yield the expected 2 bands. The efficiency of CRISPR/Cas9 can vary from 20-90% given the type of cell line used, so the results are consistent with these previous findings (Zheng Q, et al. 2014 Precise gene deletion and replacement using the CRISPR/Cas9 system in human cells. BioTechniques 57(3):115-124). These results suggest that the upper band is ZIC2 also and that the single band clones are heterozygous mutants. Lanes 5 and 7 indicate homozygous mutants and the levels of actin are also low in these lanes. These results were generated from proteins harvested by direct lysing of cells with loading buffer in 24 well plates after plating equal amounts of each clone in the wells and growing for an equal amount of time (˜3 days). The lower amount of total protein in the lines in lanes 5 and 7 suggests that these cells proliferate much slower than the other lines with one or two ZIC2 bands. This is evidence indicating that the alteration of ZIC2 levels may lead to lower proliferation rates in homozygous mutant lines. The CRISPR/Cas9 mutagenesis induces frame shift mutations leading to truncated or alternative reading frame proteins following the site of the targeting guide RNA. As state previously, the RabMab anti-ZIC2 antibody was generated against amino acids 100-130 of ZIC2, which is downstream of the anticipated gRNA mutagenesis sites. Therefore loss of protein detection is expected if the mutagenesis was successful. These results suggest that ZIC2 mutant cell lines can be generated using the methods described herein and that the antibody identified herein is specific against ZIC2. The potential homozygous loss of both of the bands observed suggests severely reduced proliferation. This is consistent with the lethality seen in homozygous ZIC2 mutant mouse lines.

The embodiments above are intended to be illustrative and not limiting. Additional embodiments are within the claims. In addition, although the present disclosure has been described with reference to particular embodiments, those skilled in the art will recognize that changes can be made in form and detail without departing from the spirit and scope of the disclosure. Any incorporation by reference of documents above is limited such that no subject matter is incorporated that is contrary to the explicit disclosure herein.

1. A cell line capable of expressing Zic family member 2 (ZIC2) protein or a mutant of ZIC2, said cell line being produced from CRISPR/Cas9 mediated genome editing.

2. The cell line of claim 1, wherein the CRISPR/Cas9 mediated genome editing comprises:
co-transfecting host cell lines with a CRISPR/Cas9 plasmid to produce the cell line,
wherein the plasmid comprising a guide RNA targeting ZIC2 gene and the guide RNA is selected from one of SEQ ID NO. 2-SEQ ID NO. 6 or a functional variant of one of SEQ ID NO. 2-SEQ ID NO. 6.

3. The cell line of claim 2, wherein the guide RNA is SEQ ID NO. 2 or a functional variant of SEQ ID NO. 2.

4. The cell line of claim 2, wherein the guide RNA is SEQ ID NO. 6 or a functional variant of SEQ ID NO. 6.

5. The cell line of claim 2, wherein the host cell line is LNcaP or PC-3.

6. The cell line claim 2, wherein the host cell line is HEK 293T having a triploid in the region of ZIC2, having the three sequences of the triploid over the region of the genome near the 118 sgRNA specified by SEQ ID NO. 9.

7. The cell line of claim 2, wherein the host cell line is a HEK 293T mutant cell line having a triploid in the region of ZIC2, having the three sequences of the triploid each over the region of the genome near the 118 sgRNA specified by SEQ ID NO. 7, 8, and 10, respectively.

8. A mutant ZIC2 protein isolated from a cell line capable of expressing Zic family member 2 (ZIC2) protein or a mutant of ZIC2, said cell line being produced from CRISPR/Cas9 mediated genome editing.

9. A method of producing a cell line capable of expressing Zic family member 2 (ZIC2) protein or mutant of ZIC2, the method co-transfecting host cell lines with a CRISPR/Cas9 plasmid to produce the cell line, wherein the plasmid comprising a guide RNA targeting ZIC2 gene.

10. The method of claim 9, wherein the guide RNA is selected from one of SEQ ID NO. 2-SEQ ID NO. 6 or a functional variant of one of SEQ ID NO. 2-SEQ ID NO. 6.

11. The method of 9, comprising inserting a guide RNA of SEQ ID NO. 2 or 6 or a functional variant of SEQ ID NO. 2 or 6.

12. The method of claim 9, comprising co-transfecting HEK 293T, LNcaP or PC-3 host cell line.

13. The method of claim 9, comprising co-transfecting a HEK 293T host cell line having a triploid in the region of ZIC2, each of the three sequences of the triploid having a sequence over the region of the genome near the 118 sgRNA specified by SEQ ID NO. 9.

14. The method of claim 13, comprising inserting a guide RNA of SEQ ID NO. 2 or 6 and co-transfecting the HEK 293T host cell line to produce a HEK 293T mutant cell line.

15. The method of claim 14, wherein the HEK 293T mutant cell line is a triploid in the region of ZIC2, having the three sequences of the triploid each over the region of the genome near the 118 sgRNA specified by SEQ ID NO. 7, 8, and 10.

16-17. (canceled)

18. A cell line capable of expressing Zic family member 2 (ZIC2) protein or mutant of the Zic2 protein, said cell line being produced from CRISPR/Cas9 mediated genome editing, wherein the CRISPR/Cas9 mediated genome editing comprising the steps of
a) inserting a guide RNA into a ZIC2 gene with CRISPR/Cas9 mediated genome editing to produce a plasmid, and
b) co-transfecting host cell lines with the plasmid to produce the cell line,
wherein the guide RNA is selected from one of SEQ ID NO. 2-SEQ ID NO. 6 or a functional variant one of SEQ ID NO. 2-SEQ ID NO. 6; and
wherein the host cell is a HEK 293T cell line having a triploid in the region of ZIC2, each of the three sequences of the triploid having a sequence over the region of the genome near the 118 sgRNA specified by SEQ ID NO. 9 or a HEK 293T mutant cell line.

19. The cell line of claim 18, wherein the HEK 293T mutant cell line is a triploid in the region of ZIC2, having the three sequences of the triploid each over the region of the genome near the 118 sgRNA specified by SEQ ID NO. 7, 8, and 10.

20. A mutant ZIC2 protein isolated from the HEK 293T mutant cell line having a triploid in the region of ZIC2, each of the three sequences of the triploid having a sequence over the region of the genome near the 118 sgRNA specified by SEQ ID NO. 9 or a HEK 293T mutant cell line.

21. The cell line of claim 5, wherein the guide RNA comprises:
SEQ ID NO. 2;
a functional variant of SEQ ID NO. 2;
SEQ ID NO. 6; or
a functional variant of SEQ ID NO. 6.

22. The cell line of claim 6, wherein the guide RNA comprises:
SEQ ID NO. 2;
a functional variant of SEQ ID NO. 2;
SEQ ID NO. 6; or
a functional variant of SEQ ID NO. 6.

23. The cell line of claim 7, wherein the guide RNA comprises:
SEQ ID NO. 2;
a functional variant of SEQ ID NO. 2;
SEQ ID NO. 6; or
a functional variant of SEQ ID NO. 6.

24. The mutant ZIC2 protein of claim 8, wherein the CRISPR/Cas9 mediated genome editing comprises:
co-transfecting host cell lines with a CRISPR/Cas9 plasmid to produce the cell line;
wherein the plasmid comprising a guide RNA targeting ZIC2 gene and the guide RNA is selected from one of SEQ ID NO. 2-SEQ ID NO. 6 or a functional variant of one of SEQ ID NO. 2-SEQ ID NO. 6.

25. The mutant ZIC2 protein of claim 24, wherein the guide RNA comprises:
SEQ ID NO. 2;
a functional variant of SEQ ID NO. 2;
SEQ ID NO. 6; or
a functional variant of SEQ ID NO. 6.

26. The mutant ZIC2 protein of claim 24, wherein the host cell line is:
LNcaP;
PC-3; or
a HEK 293T mutant cell line having a triploid in the region of ZIC2, having the three sequences of the triploid each over the region of the genome near the 118 sgRNA specified by SEQ ID NO. 7, 8, and 10, respectively.

27. The method of claim 12, wherein the guide RNA is selected from one of SEQ ID NO. 2-SEQ ID NO. 6 or a functional variant of one of SEQ ID NO. 2-SEQ ID NO. 6.

28. The method of claim 12, comprising inserting a guide RNA of SEQ ID NO. 2 or 6 or a functional variant of SEQ ID NO. 2 or 6.

29. The method of claim 13, wherein the guide RNA is selected from:
SEQ ID NO. 2-SEQ ID NO. 6; or
a functional variant of one of SEQ ID NO. 2-SEQ ID NO. 6.

30. The method of claim 29, comprising inserting a guide RNA of SEQ ID NO. 2 or 6 and co-transfecting the HEK 293T host cell line to produce a HEK 293T mutant cell line.

31. The method of claim 30, comprising inserting a guide RNA of:
SEQ ID NO. 2
SEQ ID NO. 6;
a functional variant of SEQ ID NO. 2; or
a functional variant of SEQ ID NO. 6.

32. The method of claim 31, comprising inserting a guide RNA of SEQ ID NO. 2 or 6 and co-transfecting the HEK 293T host cell line to produce a HEK 293T mutant cell line.

33. The method of claim 30, wherein the HEK 293T mutant cell line is a triploid in the region of ZIC2, having the three sequences of the triploid each over the region of the genome near the 118 sgRNA specified by SEQ ID NO. 7, 8, and 10.

33. The method of claim 32, wherein the HEK 293T mutant cell line is a triploid in the region of ZIC2, having the three sequences of the triploid each over the region of the genome near the 118 sgRNA specified by SEQ ID NO. 7, 8, and 10.

34. The cell line of claim 20, wherein the HEK 293T mutant cell line is a triploid in the region of ZIC2, having the three sequences of the triploid each over the region of the genome near the 118 sgRNA specified by SEQ ID NO. 7, 8, and 10.

35. The mutant ZIC2 protein of having a triploid in the region of ZIC2, wherein:
each of the three sequences of the triploid has a sequence over the region of the genome near the 118 sgRNA specified by SEQ ID NO. 9 or a HEK 293T mutant cell line; and
the HEK 293T mutant cell line is a triploid in the region of ZIC2, having the three sequences of the triploid each over the region of the genome near the 118 sgRNA specified by SEQ ID NO. 7, 8, and 10.


SEQUENCE LISTING (10 sequences)

SEQ ID NO: 1 - DNA, 20 bp, Artificial Sequence
  ZIC2
ccgcgcacatgggagccttc

SEQ ID NO: 2 - DNA, 20 bp, Artificial Sequence
  ZIC2
cggacccgcgtccaggagca

SEQ ID NO: 3 - DNA, 20 bp, Artificial Sequence
  ZIC2
ccgcggcggagtggtgatgg

SEQ ID NO: 4 - DNA, 20 bp, Artificial Sequence
  ZIC2
ttcacggtcctgcatctcgg

SEQ ID NO: 5 - DNA, 20 bp, Artificial Sequence
  ZIC2
agcctggcggcggcgcagaa

SEQ ID NO: 6 - DNA, 20 bp, Artificial Sequence
  ZIC2
gaaggctcccatgtgcgcgg

SEQ ID NO: 7 - DNA, 102 bp, Artificial Sequence
  ZIC2
gtgaactgagcctggcggcggcgcagaacggcttcgttgactccgccaccgaccgcacatgggagccttcaagctcaacccgtgcgcgcacgagctgtcccc

SEQ ID NO: 8 - DNA, 101 bp, Artificial Sequence
  ZIC2
gtgaactgagcctggcggcggcgcagaacggcttcgttgactccgccgccgtcgcacatgggagccttcaagctcaacccgggcgcgcacgagctgtcccc

SEQ ID NO: 9 - DNA, 100 bp, Artificial Sequence
  ZIC2
gtgaactgagcctggcggcggcgcagaacggcttcgttgactccgccgccgcgcacatgggagccttcaagctcaacccgggcgcgcacgagctgtcccc

SEQ ID NO: 10 - DNA, 92 bp, Artificial Sequence
  ZIC2
gtgaactgagcctggcggcggcgcagaacggcttcgttgactccgcacatgggagccttcaagctcaacccgggcgcgcacgagctgtcccc